(19) United States
(12) Patent Application Publication
(10) Pub. No.: US 2023/0346221 A1
(43) Pub. Date: Nov. 2, 2023

(54) METHOD, APPARATUS, AND SYTEM FOR PROVIDING GROUP SYNCHRONY BASED ON BIOMARKERS

(71) Applicant: Syneurgy, Inc., Beverly Hills, CA (US)

(72) Inventors: Shijia GENG, Beijing (CN); Erwin VALENCIA, Beverly Hills, CA (US); Michael MANNINO, Miami, FL (US); David NGO, Dallas, TX (US)

(21) Appl. No.: 18/128,110

(22) Filed: Mar. 29, 2023

(30) Foreign Application Priority Data
Apr. 29, 2022 (CN) 202210472911.9

(51) Int. Cl. A61B 5/00; H04L 67/104

(52) U.S. Cl. CPC A61B 5/0024; A61B 5/6801; A61B 5/0022; H04L 67/1044

(57) ABSTRACT

An approach is provided for group synchrony based on biomarkers. The approach involves receiving biomarker data collected from one or more sensors for a group of at least two users, wherein the biomarker data includes respective biomarker values for the at least two users, another group of other users, or a combination thereof. The approach also involves computing a group synchrony score based on a metric indicating a similarity or a difference of the respective biomarker values. The approach further involves providing the group synchrony score as an output in a user interface of a device.

### RELATED APPLICATION

This application claims priority from Chinese Patent Application Serial No. 202210472911.9, entitled “METHOD, APPARATUS, AND SYSTEM FOR PROVIDING GROUP SYNCRHRONY BASED ON BIOMARKERS,” filed on Apr. 29, 2022, the contents of which are hereby incorporated herein in its entirety by this reference.

### FIELD OF THE INVENTION

The present invention relates to a method, apparatus, and system for measurement and quantitative analysis of group physiological and/or behavioral synchrony.

### BACKGROUND

In many group activities, achieving physiological and/or behavioral synchrony among members of the group can often lead to increased group performance. However, determining when such synchrony is achieved and to what extent can be difficult. As a result, service providers face significant technical challenges with respect to automatically detecting, maintaining, and/or encouraging groups to achieve a synchronous state (e.g., physiological and/or behavioral synchrony).

### SOME EXAMPLE EMBODIMENTS

Therefore, there is a need for an approach for providing group synchrony based on biomarkers collected from group members.

According to one embodiment, a method comprises receiving biomarker data collected from one or more sensors for a group of at least two users. The biomarker data includes respective biomarker values for the at least two users, another group of other users, or a combination thereof. The method also comprises computing a group synchrony score based on a metric indicating a similarity or a difference of the respective biomarker values. The method further comprises providing the group synchrony score as an output in a user interface of a device. In one embodiment, the output can be used for monitoring progress towards achieving group synchrony, recommending actions to achieve group synchrony, providing feedback on achieving group synchrony, and/or the like.

According to another embodiment, an apparatus comprising at least one processor, and at least one memory including computer program code, the at least one memory and the computer program code configured to, with the at least one processor, cause, at least in part, the apparatus to receive biomarker data collected from one or more sensors for a group of at least two users. The biomarker data includes respective biomarker values for the at least two users, another group of other users, or a combination thereof. The apparatus is also caused to compute a group synchrony score based on a metric indicating a similarity or a difference of the respective biomarker values. The apparatus is further caused to provide the group synchrony score as an output in a user interface of a device.

According to another embodiment, a computer-readable storage medium carrying one or more sequences of one or more instructions which, when executed by one or more processors, cause, at least in part, an apparatus to receive biomarker data collected from one or more sensors for a group of at least two users. The biomarker data includes respective biomarker values for the at least two users, another group of other users, or a combination thereof. The apparatus is also caused to compute a group synchrony score based on a metric indicating a similarity or a difference of the respective biomarker values. The apparatus is further caused to provide the group synchrony score as an output in a user interface of a device.

According to another embodiment, an apparatus comprises means for receiving biomarker data collected from one or more sensors for a group of at least two users. The biomarker data includes respective biomarker values for the at least two users, another group of other users, or a combination thereof. The apparatus also comprises means for computing a group synchrony score based on a metric indicating a similarity or a difference of the respective biomarker values. The apparatus further comprises means for providing the group synchrony score as an output in a user interface of a device.

In addition, for various example embodiments of the invention, the following is applicable: a method comprising facilitating a processing of and/or processing (1) data and/or (2) information and/or (3) at least one signal, the (1) data and/or (2) information and/or (3) at least one signal based, at least in part, on (including derived at least in part from) any one or any combination of methods (or processes) disclosed in this application as relevant to any embodiment of the invention.

For various example embodiments of the invention, the following is also applicable: a method comprising facilitating access to at least one interface configured to allow access to at least one service, the at least one service configured to perform any one or any combination of network or service provider methods (or processes) disclosed in this application.

For various example embodiments of the invention, the following is also applicable: a method comprising facilitating creating and/or facilitating modifying (1) at least one device user interface element and/or (2) at least one device user interface functionality, the (1) at least one device user interface element and/or (2) at least one device user interface functionality based, at least in part, on data and/or information resulting from one or any combination of methods or processes disclosed in this application as relevant to any embodiment of the invention, and/or at least one signal resulting from one or any combination of methods (or processes) disclosed in this application as relevant to any embodiment of the invention.

For various example embodiments of the invention, the following is also applicable: a method comprising creating and/or modifying (1) at least one device user interface element and/or (2) at least one device user interface functionality, the (1) at least one device user interface element and/or (2) at least one device user interface functionality based at least in part on data and/or information resulting from one or any combination of methods (or processes) disclosed in this application as relevant to any embodiment of the invention, and/or at least one signal resulting from one or any combination of methods (or processes) disclosed in this application as relevant to any embodiment of the invention.

In various example embodiments, the methods (or processes) can be accomplished on the service provider side or on the mobile device side or in any shared way between service provider and mobile device with actions being performed on both sides.

For various example embodiments, the following is applicable: An apparatus comprising means for performing the method of any of originally filed claims 1-10, 21-30, and 46-48.

Still other aspects, features, and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The embodiments of the invention are illustrated by way of example, and not by way of limitation, in the figures of the accompanying drawings:

FIG. 1 is a diagram of a system capable of providing group synchronization based on biomarkers, according to an example embodiment;

FIG. 2 is a diagram of collaboration cycle for use with group synchronization, according to an example embodiment;

FIG. 3 is a diagram of components of a group synchronization system, according to an example embodiment;

FIG. 4 is a diagram of a technical stack for implementing a group synchronization system, according to an example embodiment;

FIG. 5 is a flowchart of a process for providing group synchronization based on biomarkers, according to one embodiment;

FIG. 6 is a diagram illustrating an example of collecting biomarker and other data for group synchronization, according to an example embodiment;

FIGS. 7A-7C are diagrams illustrating example user interfaces for providing group synchronization based on biomarkers, according to various example embodiments;

FIG. 8 is a diagram of hardware that can be used to implement an example embodiment;

FIG. 9 is a diagram of a chip set that can be used to implement an example embodiment; and

FIG. 10 is a diagram of a mobile terminal (e.g., handset) that can be used to implement an example embodiment.

### DESCRIPTION OF SOME EMBODIMENTS

Examples of a method, apparatus, and computer program for providing context-aware control of sensors and sensor data are disclosed. In the following description, for the purposes of explanation, numerous specific details are set forth in order to provide a thorough understanding of the embodiments of the invention. It is apparent, however, to one skilled in the art that the embodiments of the invention may be practiced without these specific details or with an equivalent arrangement. In other instances, well-known structures and devices are shown in block diagram form in order to avoid unnecessarily obscuring the embodiments of the invention.

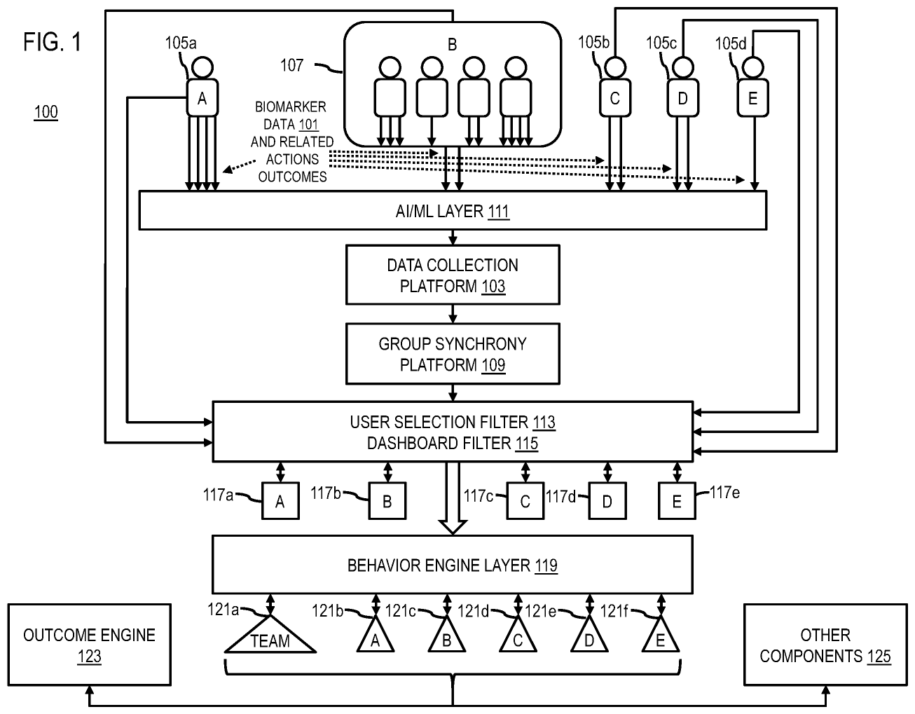

FIG. 1 is a diagram of a system 100 capable of providing group synchronization based on biomarkers, according to an example embodiment. The various embodiments described herein relate to the subject of interpersonal synchronization, specifically physiological and behavioral synchrony, and improved connection between two or more people. More specifically, it relates to using various psychophysiological biomarkers as measures for when two or more people are synchronized. As used herein, the term “synchrony” or “synchronization” refers to a psychophysiological state in which the measured biomarkers or two or more people are within threshold criteria for being similar or in phase. For example, synchrony can refer to a state when heart rates, breathing rates, and/or any other biomarker match within a threshold range. Other examples include, but are limited to, shared or mimicked behaviors between the two or more people (e.g., making similar movements, gestures, facial expressions, speech patterns, etc.).

When interacting in various ways, human beings have a natural tendency to synchronize their behaviors, gestures, physiology, and neurobiology, including breathing, heart rate, and skin conductance, as well as brain waves. In addition, it has been shown that when humans synchronize such attributes, they have better interactions and social connections, as well as increased empathy, trust, pro-sociality, generosity, wellness, cooperation, collaboration, and performance. However, detecting and quantifying when a group or team of two or more people are in synchrony presents significant technical challenges.

To address these technical challenges, various embodiments of the system 100 of FIG. 1 introduces a capability to collect various biomarkers (e.g., biomarker data 101 including but not limited to data indicating heart rate, respiratory rate, electroencephalogram (EEG), galvanic skin response (GSR), functional near-infrared spectroscopy (fNIRS), accelerometer data, any other equivalent neuroimaging techniques, any other sensor data indicative of a physiological state or condition, and/or the like collected via a data collection platform 103) and other social, environmental and ecological data (such as facial expressions, movement, contextual speech and conversation, verbal and non-verbal cues, etc.), from multiple individuals or subjects (e.g., individual users 105a-105d— also collectively referred to as individuals 105—of a group/team as well as subgroups/substreams 105 comprising multiple individuals within the larger group/team) to measure (e.g., via sensor devices associated with respective individual users 105 and/or subgroups 107) and compute a group synchrony score (e.g., via one or more algorithms of a group synchrony platform 107) as an quantitative indicator of the level of group synchrony. In one embodiment, the system 100 provides the group synchrony score as an output to individual users in a novel feedback user interface/user experience.

By way of example, in organizations and institutions that rely on team performance, as well as situations that require human collaboration, a need exists for novel methods to improve team outcomes. As such, in one embodiment, the group synchrony score enables users to identify and understand the key components of successful group/social interactions and/or any other group activities, based on specific patterns and relationships in biomarker data 101 and related data collected on what actions were performed and what outcomes (e.g., improved collaboration, increased group performance on a task, achieve mastery of group skills, etc.) were achieved by the group (e.g., by the individuals 105 and/or subgroups 107). The system 100, for instance, can include an artificial intelligence (AI)/machine learning (ML) layer 111 to automatically learn these patterns and relationships to make predictions such as but not limited to: (1) future or potential group synchrony scores; (2) future outcomes based on input biomarker data and/or group actions that are being taken; (3) recommended actions or behaviors to change the group synchrony score; and/or the like.

In one embodiment, the AI/ML layer 111 includes one or more AI/machine learning models which predict group/team performance from physiological data (e.g., biomarker data 101) during a group/team interaction. The machine learning models can be trained to make the predictions using training data (e.g., biomarker data 101 and/or related other environmental/contextual) annotated with known group/team performance outcomes. In one embodiment, the training data can be extracted from a database or data warehouse including team/group physiological data (e.g., encrypted for privacy). In another embodiment, the training data can also include video and/or audio recording data captured during group/team interactions. Then, data such as but not limited to facial expressions, movements, speech or conversations, and other verbal/non-verbal cues can be extracted as features for training the AI/ML model. In this way, the AI/ML model can predict team performance from video and audio recording during team interaction in addition to or in place biomarker data 101.

In one embodiment, to perform model training, the AI/ML layer 111 can incorporate a learning model (e.g., logistic regression model, Random Forest model, and/or any equivalent model) to train AI/ML model to make group synchrony-related predictions (e.g., outputs) from input features or signals (e.g., biomarker data 101 and/or features extracted from other data sources such as video and audio samples as described above). During training, the AI/ML layer 119 can use a learner module that feeds feature sets from a training data set into the AI/ML model to compute a predicted matching feature (e.g., group/team performance or outcome) using an initial set of model parameters. The learner module then compares the predicted matching probability and the predicted feature to ground truth data in the training data set for each observation used for training. The learner module then computes an accuracy of the predictions (e.g., via a loss function) for the initial set of model parameters. If the accuracy or level of performance does not meet a threshold or configured level, the learner module incrementally adjusts the model parameters until the model generates predictions at a desired or configured level of accuracy with respect to the annotated labels in the training data (e.g., the ground truth data). In other words, a “trained” AI/ML model has model parameters adjusted to make accurate predictions with respect to the training data set. In the case of a neural network, the model paraments can include, but are not limited, to the coefficients or weights assigned to each connection between neurons of the neural network.

As shown in FIG. 1 (via multiple arrows flowing from individuals 105 and subgroup 107), the collected biomarker data 101 can be multi-dimensional (e.g., comprised of streams of different types of biomarker data such as heartrate, respiration rate, EEG, movement, etc. with each arrow representing a different stream). Accordingly, in one embodiment, the system 100 enables each individual 105, each subgroup 107, and/or the entire team as a whole to select which streams (e.g., which biomarker data type) to collect via user selection filter 113. For example, individual user 105a (labeled “A”) has selected four different data streams or biomarker data types to collect (e.g., as indicated by 4 arrows flowing from individual 105a). Similarly, individuals 105b (labeled “C”) and 105c (labeled “D”) have selected two streams, and individual 105d (labeled “E”) has selected one stream. For subgroup 107 (labeled “B”), each individual in the group can select different streams or numbers of streams (e.g., from 1 to 4 respective for each subgroup member) to collect, and then the subgraph 107 can specify the data streams (e.g., two streams) to collect for the subgroup as a whole. The user selection filter 113 will then use the individual and subgroup selections to determine what streams of the biomarker data 101 based on the user selections to collect for generating the group synchrony score.

In one embodiment, individual users 105 and subgroups 107 can determine what data or outputs are provided in respective system user interfaces (e.g., a data dashboard user interface) using a dashboard filter 115. The dashboard or user interface can present different types of outputs (e.g., synchrony score, recommended actions, synchrony monitoring results, etc.) to display and at what level of granularity (e.g., outputs specific to an individual, subgroup, or entire team) as selected using the dashboard filter 115. In this way, each dashboard user interface (UI) 117a-117e (e.g., labeled “A”-“E”) can be independently and respectively presented to the individuals 105 and subgroups 107 who are members of the larger group of interest. By way of example, each dashboard can present the group synchrony score, the respective user's personal biomarker data 101 or contribution to the group synchrony score, and/or other related information on an individual or group basis (e.g., based on the selections in the dashboard filter 115). Thus, the dashboard UIs 117a-117e or other dashboard outputs can reflect individual, subgroup/subteam, and/or group/team selections.

In one embodiment, the outputs of the dashboard filter 115 can be feed to a behavior engine layer 119 to generate recommended behaviors and/or actions. More specifically, the behavior engine layer 119 can determine what specific actions or types of behaviors 121a-121f (also collectively referred to as behaviors 121) that the individuals 105 (e.g., behavior 121a “A” for individual 105a, behavior 121d “C” for individual 105b, behavior 121e “D” for individual 105c, and behavior 121f “E” for individual 105d), subgroup 107 (e.g., subgroup behavior 121c), and/or entire team (e.g., behavior 121a “TEAM”) can engage in to change the group synchrony score. The change can be a positive change to increase group synchrony (e.g., achieve greater alignment or similarity of biomarker signals among the members of the group) or to decrease group synchrony (e.g., achieve greater divergence or difference of biomarker signals among members of the group). Whether increased or decreased group synchrony is recommended can be based on the target outcome and/or group activity specified by the individuals 105, subgroups 107, and/or entire team. Thus, in one embodiment, the behavior engine layer 119 outputs personalized recommendations to individual and group members on the platform in real time, asynchronously, and scheduled. The personalized recommendations include, but are not limited to, prompts for users to do specific actions, insights and feedback.

In one embodiment, the recommended actions or behaviors 121 output from the behavior engine layer can be fed to an outcome engine 123. The outcome engine 123, for instance, can then monitor the biomarker data 101 collected from the individuals 105, subgroup 107, and/or entire group/team during or after the completion of the recommended behaviors 121 to determine what outcome or effect on the group synchrony score that performing the recommended behaviors 121 by the group members will have. In addition or alternatively, the outcome engine 123 can monitor when the group synchrony score reaches certain target values (e.g., a specified target score, a minimum score, a maximum score, etc.) and provide a feedback user interface to the group members about when those targets are met or about progress towards meeting those targets.

In another embodiment, the outputs of the system 100 (e.g., group synchrony score, actions/behaviors 121, and/or any other outputs) can be fed to other parties/users, services, applications, content providers, etc.

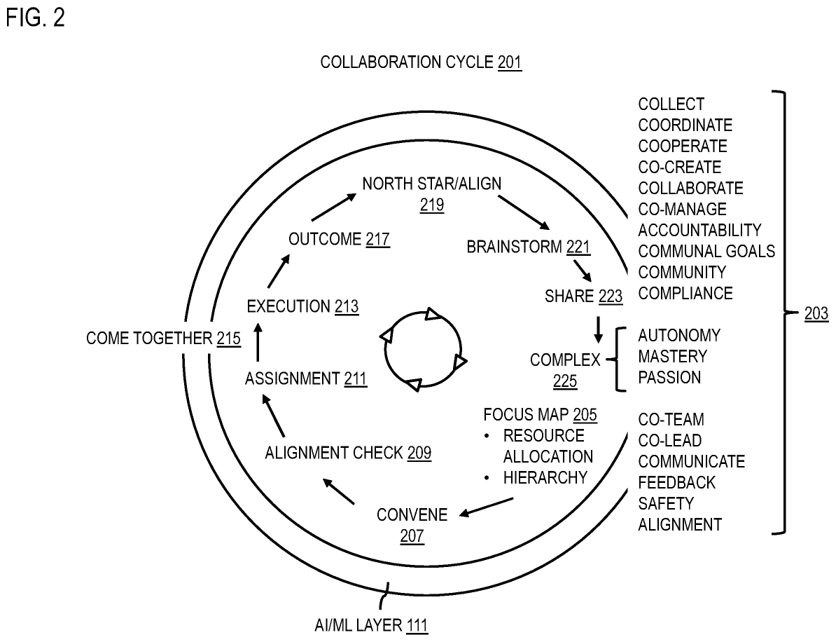

As noted above, the various embodiments described herein for quantifying a group synchrony score based on biomarker data 101 can be used for assessing or improving group activities such as but not limited to group collaboration. FIG. 2 is a diagram of collaboration cycle 201 for use with group synchronization, according to an example embodiment. In the example of FIG. 2, the collaboration cycle is associated with various action/outcome labels 203 such as but not limited to: collect, coordinate, cooperate, co-create, collaborate, co-manage, accountability, communal goals, community, compliance, autonomy, mastery, passion, co-team, co-lead, communicate, feedback, safety, and alignment. The examples listed above are provided by way of illustration and not as limitations. It is contemplated that the action/outcome labels 203 can include any action that can be taken as part of the collaboration cycle 201 and any outcome (e.g., intermediate or final outcome) that can be achieved using the collaboration cycle 201. The specific labels 203, for instance, can depend on the type of collaborative activity being performed (e.g., team sports, business group, school team project, etc.).

Regardless of the specific type of collaborative activity, the collaboration cycle 201 can typically start with a focus map activity 205 during which the group will determine a resource allocation (e.g., determine group members and what resources are available to those group members) and a group hierarchy (e.g., determine roles for group members such as but not limited to co-team and co-lead roles) and how the group will interact (e.g., how to communicate, ensure safety, ensure alignment of group efforts towards a common goal). Next, the group will convene at 207 and perform an alignment check 209. The alignment check 209, for instance, ensures that the group members are in a state that will enable them to achieve the goals of the collaboration cycle 201. In one embodiment, the state can be measured using the various embodiments of the group synchrony score based on biomarker data 101 described herein.

Based on this alignment check 209, the task assignments 211 can be given to members of the group for execution 213 after coming together 215 for the collaborative task. After execution 213, the outcome 217 of the collaborative task can be assessed (e.g., successfully completed, failed, partially completed, etc.). The outcome 217 can then be used to again assess the alignment 219 of the group members towards a common goal or “north star.” Based on the outcome 217 and alignment 219, the group members can brainstorm 221 to determine new ideas or actions to continue the collaboration or to start a new collaboration and then to share the ideas with group members and other stakeholders at 223. The collaboration cycle 201 can continue until as set group outcome (e.g., autonomy, mastery, passion, etc.) is achieved with respect to the collaborative task.

As illustrate in this example, the collaboration cycle 201 can be a complex process 225 involving many states and action/outcome labels 203 that often are not amenable to be technological solutions. In one embodiment, the system 100 address this challenge by computing the group synchrony score at one or more of the steps 205-225 of the collaboration and then using the AI/ML layer 111 to learn the relationships between group synchrony scores, corresponding collaborative steps 205-225 (or other related actions), and outcomes 217 (e.g., success or failure of a collaborative step or action to achieve a state goal). For example, ground truth data can be collected of group synchrony scores that are associated with known collaborative steps 205-225 and known outcomes 217 can be gathered to train an AI/ML model (e.g., a neural network or equivalent) of the AI/ML layer 111 to encode those relationships in the trained AI/ML model. This trained AI/ML model will then be able to predict what actions to perform to achieve a certain group synchrony score or a certain outcome, or to predict other permutations of the relationship such as but not limited to what outcome or action can be achieved given a certain group synchrony score. Thus, in one embodiment, the AI/ML model can be trained using any combination of the parameters of group synchrony score, actions, and outcomes as inputs to predict any other combination of the parameters as outputs of the AI/ML model.

For example, the embodiments of the system 100 can be used to predict what group synchrony score is best for a brainstorming session (e.g., greater than a certain number of actionable brainstorming ideas can be used as a measure of a successful outcome at brainstorming step 221) versus what group synchrony score is best for task execution (e.g., execution step 213 resulting in a task completed on time, on budget, with a target level of quality, etc.). The machine-learned relationships may indicate that a low group synchrony score is needed for successful brainstorming to get a diversity of ideas while a high group synchrony score is needed for group members to focus with common purpose on completing a task).

Thus, the various embodiments of the system 100 described herein provide a novel application of collected biometric and individual data (e.g., biomarker data 101) for use in measuring and performing quantitative analysis/prediction of group psychophysiology with respect to collaborative or team tasks/activities.

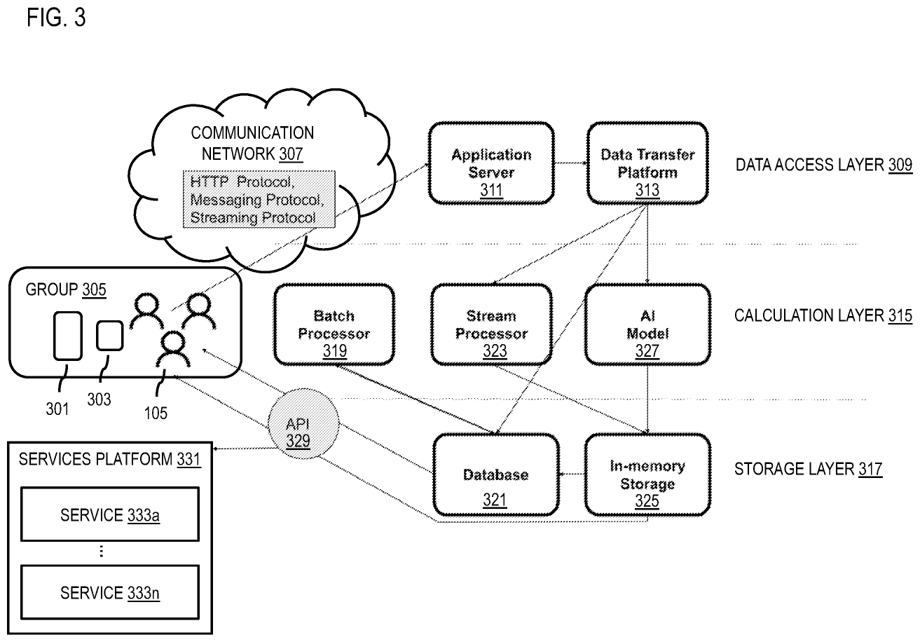

FIG. 3 is a diagram of components of a group synchronization system 100, according to an example embodiment. As shown in FIG. 3, the group synchronization system 100 includes one or more components for group synchronization based on biomarker data 101 according to the various embodiments described herein. It is contemplated that the functions of the components of the system 100 may be combined or performed by other components of equivalent functionality. In one embodiment, the system 100 includes one or more user equipment (UE) devices 301 (e.g., smartphones, laptops, computers, etc.) that are equipped with or otherwise have connectivity to one or more sensors 303 for collecting biomarker data 101 from a group 305 of individual users 105. The sensors 303, for instance, can include any type of biomarker sensor including but not limited to heart rate sensors, respiration sensors, EEG sensors, GSR sensors, fNIRS sensors, and/or the like as well as camera sensors, microphones, movement sensors, etc. to capture facial expressions, movements, speech and conversations, verbal and non-verbal cues, etc. associated with the users 105.

The UE devices 301 can transmit biomarker data 101 collected using the sensors 303 over a communication network 307 (e.g., supporting communication protocols such as but not limited to HTTP protocol, any messaging protocol, any steaming protocol, or equivalent) to the data access layer 309 for processing. As shown, the data access layer 309 comprises an application server 311 for executing one or more applications accessible via the UE devices 301 (e.g., executing client applications) for collecting biomarker data 10 and providing user interfaces for interacting with group synchronization functions (e.g., user selection filter 113 and/or dashboard filter 115). The application server 311 interacts with the data transfer platform 313 to collect and distribute biomarker data 101 from the group 305 to other layers of the system 100 (e.g., calculation layer 315 and storage layer 317). In one embodiment, the data access layer 309 is the data collection platform 103 of FIG. 1 or at least a part of the data collection platform 103.

In one embodiment, the data access layer 309 transmit collected biomarker data 101 to the calculation layer 315 for processing. As shown, the calculation layer comprises: (1) a batch processor 319 for offline or batching processing of biomarker data 101 that has aggregated and stored in a database 321 of the storage layer 316; (2) a stream processor 323 for online or real-time processing of biomarker data 101 that is transmitted directly from the data transfer platform 313 or aggregated in in-memory storage 325 of the storage layer 317; and (3) an AI model 327 (e.g., a neural network, support vector machine, random forest, decision tree, or equivalent) for processing the biomarker data 101 and related actions and outcomes to make predictions or extract features for processing by the batch processor 319 and/or stream processor (e.g., using one or more transformation algorithms to generate a group synchrony score and/or related analyses/monitoring/recommendations) according to the various embodiments described herein. In one embodiment, the batch processor 319 and stream processor 323 form the group synchrony platform 109 of FIG. 1 or are at least part of the group synchrony platform 109. In one embodiment, the AI model 327 is the AI/ML layer 111 of FIG. 1 or at least part of the AI/ML layer 111.

In one embodiment, the group synchronization system 100 provides access to its functions and/or outputs via an application programming interface (API) 329. By way of example, the API 327 provides software interfaces to external services, applications, etc. that can use or provide data for generating the group synchrony data output (e.g., group synchrony scores, predictions, recommended actions/behaviors, etc.) of the system 100. Access to the API 329 can be provided to the community in general to specific services or applications. Example of these external services include but is not limited to a services platform 331 comprising one or more services 333a-333n that use, rely on, or provide data for generating the outputs of the group synchronization system 100.

In one example use case, the services platform 331 and/or services 333 that use the outputs (e.g., behavior designs) of the group synchrony system 100 can include, but not limited to, service provider systems that facilitate workshops to help client groups/teams define their team behavior goals and create custom programs/packages for client teams to increase collaboration and business objectives (e.g., such as improvements reaching target group synchrony states during the collaboration cycle 201). For example, the services 333 can use the system outputs as part of a behavior design process that shapes group behavior, actions, outcomes, etc. using monitored group synchrony scores to improve product innovation, customer experience design, service delivery, and/or the like.

Though depicted as separate entities in FIG. 3, it is contemplated that the any of components of the group synchrony system 100 may be implemented as a module of any other equivalent component. In another embodiment, one or more of the components of the system 100 may be implemented as a cloud-based service, local service, native application, or combination thereof. The functions of the group synchrony system 100 and its components are discussed with respect to figures described below.

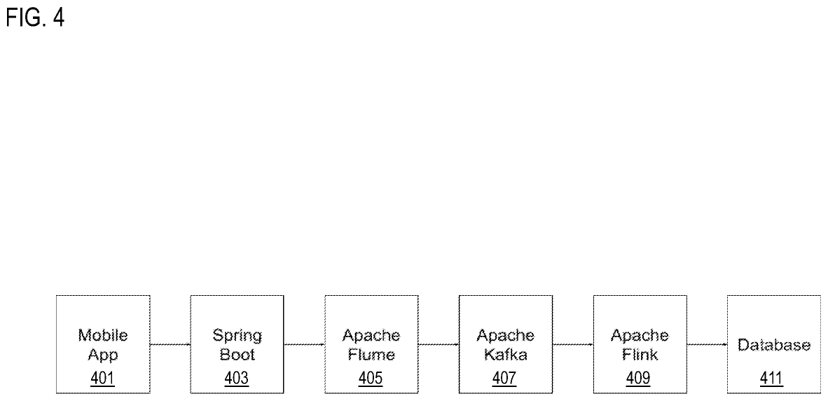

FIG. 4 is a diagram of a technical stack for implementing the group synchronization system 100, according to an example embodiment. As shown, a mobile application 401 (e.g., executing on a UE device 301) is created to act as a client for collecting biomarker data 101 and presenting a dashboard or other user interface for presenting the output of the group synchronization system 100. The mobile application 401 can also provide a user interface for interacting with the user selection filter 113 and/or dashboard filter 115. In one embodiment, the mobile application 401 can be built based on a Spring Boot framework 403 (or equivalent). Spring Boot, for instance, is a Java-based framework to create applications. This Spring Boot framework 403 interfaces with a biomarker data collection and aggregation pipeline. One example (but not exclusive example) of this pipeline comprises: (1) an Apache Flume component 405 (or equivalent) for efficiently collecting and aggregating large amounts of biomarker data 101; (2) an Apache Kafka component 407 (or equivalent) for streaming the biomarker data 101 collected by the Apache Flume component 405; and (3) an Apache Flink component 409 (or equivalent) for providing stateful computations over the biomarker data stream generated by the Apache Kafka component 407. This pipeline takes the raw biomarker data 101 collected via the Mobile Application 401 and transforms them into a format and structure that can be processed by the processing components (e.g., calculation layer 315) of the system 100, for storage in the database 411 (e.g., the storage layer 417 comprising the database 421 and in-memory storage 325).

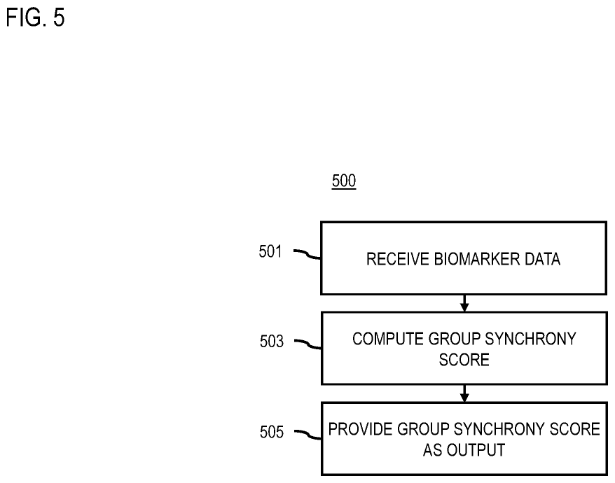

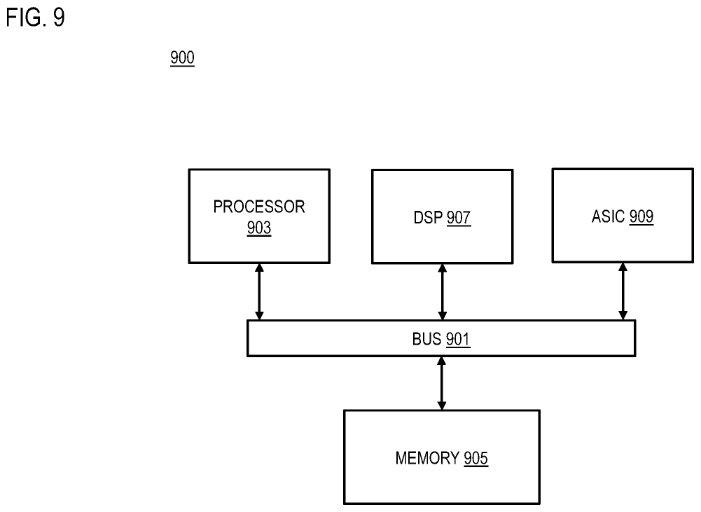

FIG. 5 is a flowchart of a process for providing group synchronization based on biomarkers, according to one embodiment. In various embodiments, the group synchronization system 100 and/or any of its components may perform one or more portions of the process 500 and may be implemented in, for instance, a chip set including a processor and a memory as shown in FIG. 9. As such, the group synchronization system 100 and/or any of its components can provide means for accomplishing various parts of the process 500, as well as means for accomplishing embodiments of other processes described herein in conjunction with other components of the system 100. Although the process 500 is illustrated and described as a sequence of steps, it is contemplated that various embodiments of the process 500 may be performed in any order or combination and need not include all of the illustrated steps.

In step 501, the data collection platform 103 receives biomarker data 101 collected from one or more sensors 303 for a group 305 of at least two users 105. In one embodiment, the biomarker data 101 includes respective biomarker values for the at least two users 105, another group of other users (e.g., a group or other subgroup against which the users 105 are to be compared for group synchrony), or a combination thereof.

In one embodiment, the data collection platform 103 is a multi-party (e.g., multiple individual users 105, subgroups 107, etc.), multi-input (e.g., multiple data biomarker sensor data streams), scaling data aggregation platform to collect available biomarker data 101 combined with available discrete and non-discrete social, environmental, ecological, and behavioral data, from endogenous or exogenous, actual or simulated sources or data sets.

In one embodiment, the data platform 103 includes an input module, funnel, or pipeline, receiving the data from any number of data sources (e.g., the example data pipeline technical stack of FIG. 4). By way of example, data sources can be any combination of real-time and pre-sorted, pre-recorded, or pre-selected endogenous (e.g., internal to the user such as biomarkers) or exogenous (e.g., external to the user such as but not limited to environment, context, etc.) data.

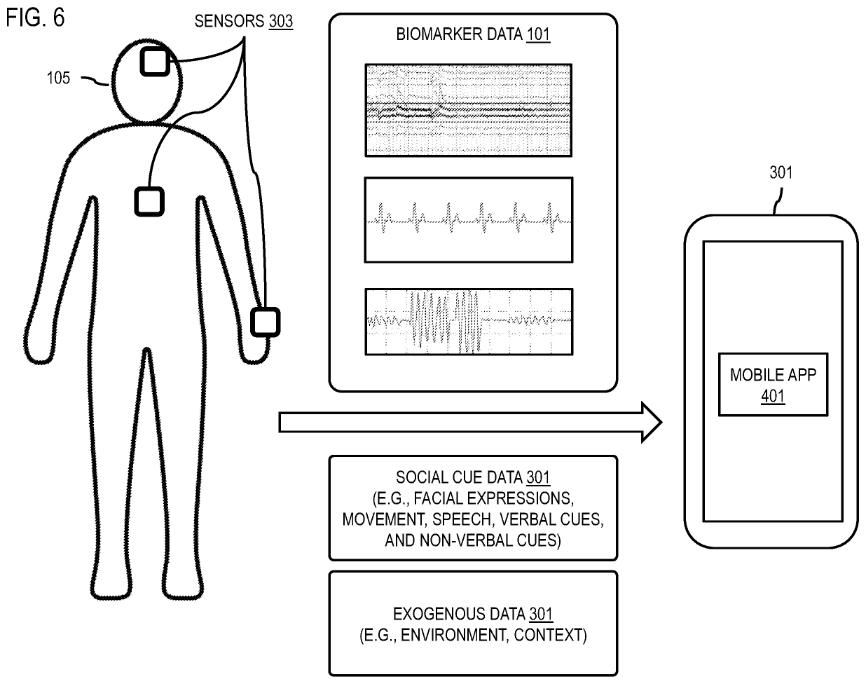

FIG. 6 is a diagram illustrating an example of collecting biomarker and other data for group synchronization, according to an example embodiment. In the example of FIG. 6, an individual user 105 is using a UE device 301 (e.g., smartphone) to executing a group synchrony mobile application 401. The mobile application 401 (or any other equivalent client device, service, application, etc.) interfaces with one or more sensors 303 associated with the individual user 105 (e.g., sensors 303 are worn, attached, external to the user 105 but with a field of view of the user 105, etc.) to capture and collect biomarker data 101 (e.g., EEG, heart rate, accelerometer, fNIRS, etc. data). The sensors 303 can also capture social cue data 601 such as but not limited to facial expressions or non-verbal cues (e.g., based on automated facial expression recognition processing of images captured by the sensors 303, and/or the like), movements (e.g., based on accelerometers, gyroscopes, automated movement recognition processing of images captured by the sensors 303, and/or the like), and speech or verbal cues (e.g., based on speed recognition processing of audio samples captured the sensors 303). In one embodiment, the sensors 303 can also collect exogenous data including but not limited to environmental factors (e.g., weather, temperature, lighting, etc.) and/or contexts (e.g., current activity, location, time of day, day of week, etc.). In yet another embodiment, the biomarker data 101 can be collected over a time frequency domain so that changes in the biomarker data 101 can be captured or otherwise determined over a designated period of time.

In one embodiment, the users 105 can use the mobile application 401 (or equivalent client) to select or otherwise use their own live data, asynchronous data, previous data, own group data, other group data, other defined data sets, undefined data sets, model data sets, etc. for generating data for the group synchrony system 100.

In one embodiment, the UE device 301, mobile application 401, or other equivalent client can include a user interface module (e.g., device based, web based, app based, xR based, etc. module) including initial query of starting state (e.g., as indicated by the user's starting biomarker readings) and intention, desired outcome, or other concrete or abstract user-selected measure of the multiparty interaction (e.g., such as the various action/outcome labels 203 of the collaboration cycle 201). In other words, the mobile application 401 can receiving an input specifying an initial state (e.g., a characterization or label representing the initial) of the group. The system 100 can then compute and generate an association between the group synchrony score and the initial state and provide this association as an output. In this way, the initial state (regardless of how abstract) can be associated with a quantified group synchrony score.

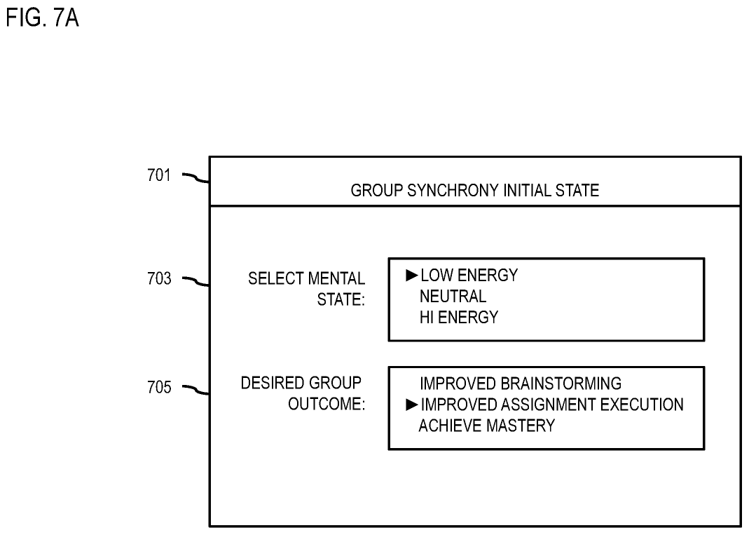

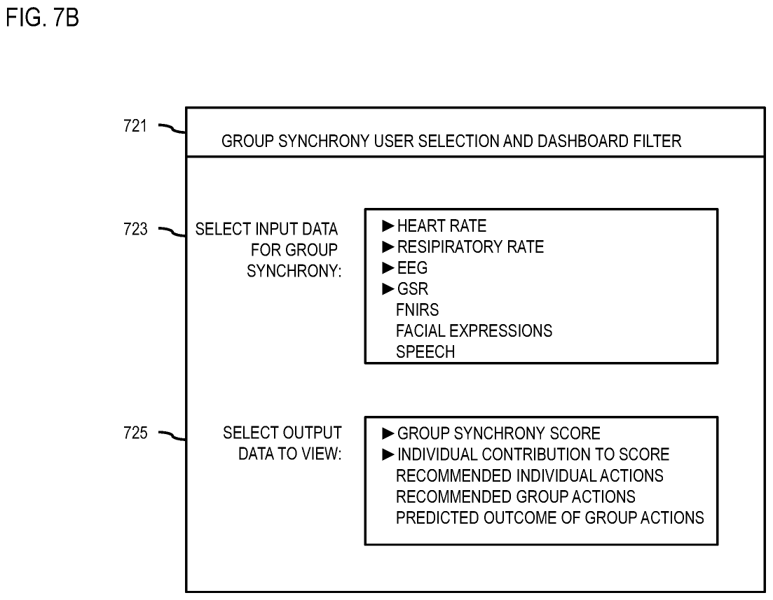

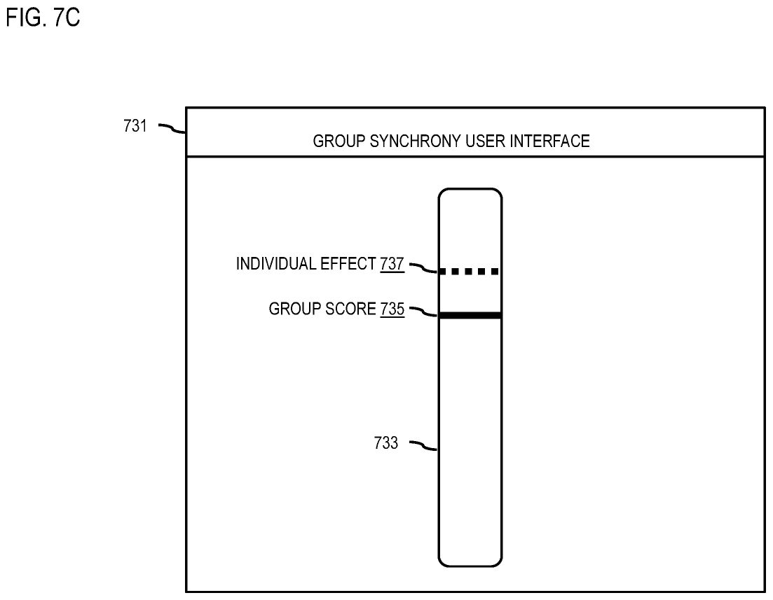

FIG. 7A illustrates an example user interface (UI) 701 for assessing the user's initial state or intention with respect to group synchrony. For example, the UI 701 (e.g., provided via the mobile application 401) asks the user or group where they are psychophysiologically, mentally, intention, etc., as well as the objective of the interaction. In this example, UI element 703 requests that the user or group select their current mental state (e.g., “Low energy,” “Neutral,” “High energy”). It is noted that the mental state label can be any term or state that used to label data for the AI/ML layer 111 to learn. Thus, potentially abstract terms are transformed into concrete labels an associated conditions or features (e.g., biomarker features) for quantification using machine learning. The UI 701 also includes a UI element 705 for specifying the desired outcome or objective of the group interaction in which the users are to be engaged. For example, UI element 705 presents labels such as but not limited to “Improved brainstorming,” “Improved assignment execution,” and “Achieve mastery.” Again, it is noted that these outcome or objective labels are provided by way of illustration and not as limitations. These labels can also be any term or label selected for AI/ML processing.

In other words, the UI element 705 can be used to receive an input specifying a target state, target interaction, or a combination thereof of the group. The system 100 can then initiate a comparison of the group synchrony score to the target state, the target interaction, or a combination thereof. This comparison, for instance, can be used as feedback output or other indicator of progress towards achieving the specified target state, target interaction, etc.

In one embodiment, the mobile application 401 (or equivalent client) can also include an individual user interface module for selection of an initial dimension of a multi-dimensional data construct within an individual user data set (e.g., collected biomarker data and other related data such as environment, context, etc.). The user interface module, e.g., enables the user to select how does the user want to see the data, what aspects of the incoming data and/or the user's relationship to that data and affect upon it. In other words, the user interface module enables the user/group to make selections for the user selection filter 113 and/or dashboard filter 115.

FIG. 7B illustrates an example UI 721 for filter selection, according to one embodiment. As shown, the UI 721 includes a UI element 723 for selecting the initial dimension (e.g., selected one or more biomarker sensor data streams) of the multi-dimensional data construct (e.g., all available biomarker sensor data streams). The selections in the UI element 723 can be used to configure the user selection filter 113. The UI 721 also includes UI element 725 for selecting the group synchrony outputs (e.g., group synchrony score, recommendation actions, and/or other predictions of the AI/ML layer 111) for presentation or monitoring in a dashboard UI. These selections can be selected with respect to outputs for specific individuals 105, subgroups 106, and/or the entire team. The selections made in the UI element 725 can be used to configure the dashboard filter 105.

In addition or as an alternate to the embodiments of the user interface module described above, the mobile application 401 (or equivalent client) can also include a group user interface module including aggregated query metric of starting state and collective intention, desired outcome, or other concrete or abstract multi-user or group leader selected measure of the collective multipart interaction which influences data delivery format. In this way, the entire group as a whole can determine how does the group want to see and interact with the data, what aspects of the incoming data and/or the group's relationship to the data and affect upon it.

In summary, as part of the data collection process, the system 100 provides via the user selection filter 113 and dashboard 115 variations of user input and feedback mechanisms that are applicable for group synchrony. For example, an individual, group, global, or platform selected feedback data and visualizations are created and suggested based on information comprised of the psychophysiological, ecological, behavior, or response data. This provides the capability to select multiple additional metrics from which to develop the group synchrony score metric and options for display the score and/or related outputs.

In one embodiment, the system 100 includes a device specific, user determined, interface for selecting preferences of data feedback (e.g., a visualization selection module such as the dashboard filter 115).

In one embodiment, the system 100 includes an individual device specific, user determined, interface for selecting preferences of data feedback that can only be observed by the user and comprised of user's own data and comprised of other group authorized data. This embodiment comprises an individual user view of the group synchrony outputs.

In one embodiment, the system 100 includes a group device specific, group determined, interface for selecting preferences of data feedback that can be observed by the group and comprised of group's own data and comprised of other group or global authorized data. This embodiment comprises a group or aggregate view of the group synchrony outputs.

In one embodiment, the system 100 includes a region and device specific, individual or group or global determined, interface for selecting preferences of data feedback that can be observed by the group and comprised of group's own data, and comprised of other group or global authorized data to quantify and to visualize metrics related to a specific or multidimensional region. This embodiment comprises a region-specific metric and view.

In one embodiment, the system 100 includes a global device specific, individual or group or global determinant, interface for selected preferences of data feedback that can be observed by the group and comprised of group's own data and comprised of other group or global authorized data. This embodiment comprises a global view.

In step 503, after collecting and manage biomarker data 101 as indicated above, the group synchrony platform 109 (e.g., the calculation layer 315) can combine the collected and aggregated data and to quantify concrete and abstract information as a multi-faceted, multi-dimensional, artifact that is referred to herein as the group synchrony score. In one embodiment, the group synchrony platform 109 can compute the group synchrony score based on a metric indicating a similarity, a difference, a priority, relative importance, etc. of the respective biomarker values in the biomarker data 101 for the individuals 105, subgroups 307, or entire group separately or in combination. In another embodiment, the group synchrony score can include a global specific, individual or group or global determinant, metric for selected preferences of data feedback that can be observed by the group and comprised of group's own data, and comprised of other group or global authorized data. The embodiment comprises a global metric based on behavior data.

For example, a data identification and sorting tool (e.g., AI/ML layer 111 or other component of the system 100) will recognize incoming data type, relative volume, and relative importance to the subjects and interaction in question (e.g., based on a pretrained AI model 327), and will prioritize that data which is most robust and contextually relevant to generate the group synchrony. For example, agile data weighting that is user selected (e.g., via user selection filter 113) and weighting that is based on what is happening in group interaction (e.g., a group call) can determined. The group synchrony platform 109 then uses the weighting to recognize what data is coming in and its importance to the intention of the call, or the relative importance to the data incoming (e.g., recognizes based on AI/ML layer 111). The group synchrony platform 109 will then automatically align to the most robust or important data stream in weighting the group synchrony score and downstream behaviors or actions.

In one embodiment, after computing the group synchrony score, a comparison tool of the group synchrony platform 109 can monitor, compare, and contrast incoming data streams with starting state intentions of the group. For example, the tool or module can monitor data and actions to determine which phase they are in, whether the group synchrony score is consistent with the intended actions or outcomes. In other words, the group synchrony platform 109 initiates a comparison of the group synchrony score, the respective biomarker values, the similarity of group biomarker data, the difference of group biomarker data, or a combination thereof to biomarker criteria associated with a reference group interaction (e.g., the group interaction in which the group members are currently involve, and/or intended outcome). For example, the group synchrony score can learn (e.g., via AI/ML layer 11) or otherwise designate a range of group synchrony scores that is associated with a given action and/or outcome (e.g., improved brainstorming, improved task execution). Thus, if the goal of the group, for instance, is to achieve “improved brainstorming” then the group synchrony score can be monitored or compared to a score range (e.g., the biomarker criteria) that is associated with the intended action/outcome. In one embodiment, biomarker criteria can include the group synchrony and/or individual target ranges for biomarker values for each different type of data stream (e.g., heart rate, EEG, GSR, etc.).

In one embodiment, the behavior engine layer 119 can determine or predict a recommended action to be performed by the group, one or more of the at least two users of the group, or a combination thereof to change the group synchrony score. For example, a behavior recognition and suggestion tool or engine will recommend actions to individuals 105, to subgroups 108, and/or to groups to align with starting state selections. In this case, the recommended action can take advantage of the current state (e.g., group synchrony score) by determining actions that are best performed while the group is already in a given group synchrony state. In this way, the behavior engine layer 119 can make adjustments to better fit existing circumstances of the group and direct the group back to a common goal/task and desired outcome. For example, the system 100 can determine a current activity in which the group is engaged (e.g., via user input or automatic detection), and then generate a prediction of whether the group will achieve the target state, target interaction, or a combination thereof based on the current activity and the group synchrony score. This prediction, for instance, can be generated using the AI/ML, layer 111 or equivalent.

In addition or alternatively, the behavior recognition and suggestion tool or engine (e.g., behavior engine layer 119) can recommend actions to individuals 105, to subgroups 108, and/or to groups to conflict with starting state selections to make adjustments to better fit existing circumstances and direct the group back to goal/task and desired outcome.

In one embodiment, the behavior recognition and suggestion tool or engine (the behavior engine layer 119) can recommend actions to individuals 105, to subgroups 108, and/or to groups to reassess the correct fit of starting state selections. For example, the starting state selections may include specific task/role assignments to individuals or include specific intents/outcomes. The behavior engine layer 119 an assess whether these initial selections result in group synchrony scores that are compatible with achieving the intents/outcomes. For example, if after performing a group task/interaction under the initial state selection, the behavior engine layer 119 determines that the task is not completed with a desired outcome (e.g., brainstorming that achieves more than a designated number of actionable ideas) and that the group synchrony score does not fall within the range associated with achieving the intent/outcome then the group synchrony platform 109 and/or engine 119 can make recommendations to make adjustments or changes to better fit circumstance and direct the group back to a common goal, task, outcome, etc.

In step 505, the group synchrony platform 109 provides the group synchrony score, recommended behaviors/actions, and/or related adjustments as an output in a user interface of a device. For example, the output can be provided via single or multiple devices and information feedback systems based on the dashboard filter 115 selections and the individual/group views discussed above in the embodiments of step 501. In addition, the group synchrony score can be computed and provided in real-time, asynchronously, or according to a schedule to update the output in the user interface.

In one embodiment, the score and/or behavior outputs can be provided to the outcome engine 123 for additional analysis. For example, the outcome engine 123 can include a behavior to outcome recognition tool comprised of a system that recognizes and projects outcomes across individuals, groups, regions with quantification and visualization of data relevant to the interaction. This embodiment comprises a region-to-region specific metric and view.

In one embodiment, the outcome engine 123 can include a multi-variable, multi-outcome recognition and comparison tool or engine that directs or suggests behaviors to promote outcomes by, for instance, enabling users or the system 100 to compare, contrast, adjust behaviors to best fit group intentions and interactions.

In one embodiment, the outcome engine 123 can include a multi-variable, multi-outcome recognition and comparison tool or engine that determines or projects outcomes based on behaviors by, for instance, enabling users or the system 100 to compare, contrast, adjust behaviors to best fit outcomes.

In one embodiment, the outcome engine 123 can include a global specific, individual or group or global determinant, metric (e.g., group synchrony score) for selected preferences of data feedback that can be observed by the group and comprised of group's own data, or comprised of other group or global authorized data for modifying behavior for outcome. In this way, a global metric based on intended outcome can be determined and monitored.

In one embodiment, the group synchrony outputs (e.g., the group synchrony score, actions/behaviors, outcomes, etc.) can be presented in a user interface using any type of UI representation. For example, the user interface can use a user or group selected graphical representation or symbol that adjusts and modifies its structure based on behaviors, projected or actual outcomes, and actual or synthetic data sets. In this way, the user interface can present visual feedback of real time individual effect on group metrics.

FIG. 7C illustrates an example group synchrony feedback UI 731 that uses a bar representation 733 of the group synchrony score with the bottom of the bar representation is a minimum score and the top of the representation a maximum score. A line 735 represents the group synchrony score for the entire group at a corresponding relative position on the bar representation 733. This group synchrony score, for instance, is computed according to the various embodiments escribed herein based biomarker data 101 collected from group members. In one embodiment, the individual effect can be computed as a deviation (e.g., standard deviation or equivalent) of an individual's biomarker values from the group biomarker values (e.g., mean, average, designated percentiles, or other equivalent statistical characterization). The computed individual effect can then be represented in the UI 731 (e.g., as dashed line 737). In this example, the positive effect on achieving group synchrony (e.g., the individual's score matches more closely to the mean, average, etc. of the overall group) is shown by placing the individual effect line 737 above the group score line 735. If there is a negative effect, the individual effect line 737 can be shown below the group score line 735. It is noted that the UI 731 is provided by way of illustration and not as a limitation. It is contemplated that any other type of representation can be made.

In one embodiment, the system 100 can present a user interface including a user or group selected graphical representation or symbol that adjusts and modifies its structure based on behaviors, projected or actual outcomes, and actual or synthetic data sets in real time. In this way, the user can receive visual feedback of real time group effect on individual metrics. For example, such real-time feedback can be provided during a group video call, so that individuals on the call can view group synchrony feedback while collaborating on the call. In another use case, each user can have individual devices (e.g., UE devices 301) to obtain individual or group synchrony feedback. If the devices are portable (e.g., smartphones, wearable devices, augmented reality devices, etc.), the feedback can be provided during any type of group interaction.

In one embodiment, the system 100 can present a user interface including a user or group selected graphical representation or symbol for observed behaviors as related to user or group selected metrics that adjusts and modifies its structure based on behaviors, projected or actual outcomes, and actual or synthetic data sets in real time. The selected metrics, for instance, are based on user filter selection 113 and/or dashboard filter 115. In this way, the system provides visual feedback of selected performance metrics.

It is contemplated that the various embodiments for group synchrony described herein can apply to a group of any size. For example, they can apply to small groups of least two parties (e.g., Coach and Client) to medium sized groups (e.g., 10+ person teams) to large group events (e.g., 1000+ people).

In addition, it is contemplated that the system 100 enables a variety of novel user experiences/user interfaces (UX/UI). For example, as discussed and illustrated in some of the example user interfaces, visual, auditory, and haptic feedback loop displayed on any type of screen (e.g., laptop, mobile app, wearable, TV, tablet, as well as augmented reality (AR)/mixed reality (MR)/virtual reality (VR), etc.) that displays individual and group synchrony/collaboration score in real-time.

In one embodiment, via the presented user interfaces, users can also see prompts and notifications of suggestions of what actions to do to increase their individual and group synchrony scores. In addition, users can also set preferences on difficulty levels with respect to achieving certain synchrony scores (e.g., challenge/skill ratio). In other words, users can be challenged to achieve a target group synchrony score while engaged in a particular activity. For example, a sports team can be challenged to achieve and maintain target group synchrony score for a designated period of time (e.g., longer periods being more difficult).

It is contemplated that any type of representation of achieving a target group synchrony score can be used. For example, a user interface may display a picture that is blurred at first and the gets clearer as the group achieves higher group synchrony scores. This visual feedback, for instance, can be an exercise. In another example, vibrations that change (e.g., increase or decrease) in proportion to the group synchrony score can be initiated on compatible devices such as, but not limited to smart watches, mobile phones, and/or any other equivalent device or peripheral. In this way, vibration motors in the devices associated with one or more group members can be actuated to convey the magnitude of group synchrony scores (e.g., either individual or group synchrony scores) to each member of the group.

In one embodiment, users can show a replay of data and sessions for individual users and/or groups along with accompany group synchrony scores determined over the same time period. These replays can be accompanied in the user interface with messages indicating analysis and recommendations for improving individual and group synchrony scores (e.g., messages indicating here is what you did and here is what you could have done to improve the group synchrony score at a particular time). More specifically, the system 100 can record the biomarker data 101 (and/or group/individual synchrony scores computed therefrom) as the group, one or more of the at least two users, or a combination thereof performs an activity. The system 100 can then replay the group synchrony score, the recorded biomarker data, or a combination thereof in association with a progress of the activity in the user interface of the device.

In one embodiment, the synchrony score(s) determined according to the various embodiments described herein can be presented in real-time during a group activity. For example, members of a sports team (or any other group) can wear haptic or vibration devices that can actuate different vibration strengths corresponding to current or real-time group synchrony scores (or an individual's contribution to the overall group synchrony score) as they engage in a sports or group activity. It is noted that vibrations through wearable haptic/vibration devices are provided by way of illustration and not as limitations to how a group synchrony scores can be presented in real time. It is contemplated that any other equivalent type of UX/UI (e.g., visual, audio, etc.) can be used to convey group synchrony scores (e.g., real-time, historical, and/or predicted scores).

In one embodiment, the group synchrony score can be used as a parameter for enabling group functions, applications, services, etc. For example, for a meeting involving a group (e.g., a video conference call), individuals participating in the meeting may be placed in virtual waiting rooms until the group synchrony score (or an individual's contribution to the group synchrony score) meets a threshold level. Then, the individuals can be admitted to the video conference call or other group activity. In some cases, the threshold level can be set to ensure that the group has at least a target level of synchrony to increase the probability of having improved teamwork. Conversely, the threshold level can be set to ensure a target level of asynchrony to increase the probability that the people with different perspectives or viewpoints participate (e.g., to foster improved brainstorming or different ideas/approaches).

In one embodiment, the system 100 can determine and monitor how well the group and/or individuals within the group can get in synchrony (e.g., achieve a target group synchrony score) or out of synchrony (e.g., the group synchrony score falls below a target level). For example, how well an individual or group can get into or out of synchrony can be determined based on the time it takes for the individual or group to increase or decrease its group score synchronize to achieve or fall below a target score.

In yet another embodiment, the user interface can be configured to enable the group and/or individual members of the group to filter out certain biomarkers, characteristics, cues, etc. from use in determining a group synchrony score or an individual's contribution to the score.

It is noted that the above use case examples are provided by way of illustration and not as limitations. As previously noted, it is contemplated that any type of UX/UI can be created to enable users to receive or interact with the functions of the group synchrony system 100.

Within the context of systems for determining group synchrony based on biomarker data 101, applicable sensors 303 include but are not limited to include heart rate sensors, respiratory rate sensors, electrocardiograph (ECG) sensors, photoplethysmograph (PPG) sensors, galvanic skin response (GSR) sensors, electroencephalograph (EEG) sensors, electromyography (EMG) sensors, neuroimaging sensors (e.g., fNIRS) and the like. In one embodiment, the sensors 113 support continuous or substantially continuous monitoring, or monitoring based on a schedule.

In one embodiment, the system 100 includes a UE device 301 with connectivity to at least one 303. In one embodiment, the sensor 303 can include but is not limited to a wearable sensor in which multiple sensors are included to provide additional functionality. In one embodiment, the UE device 301 can include the sensors 303 or have connectivity to standalone sensors 303 that can operate independently or in coordination with the UE 301. By way of example, connectivity between the UE device 301 the sensors 303 can be facilitated by short range wireless communications (e.g., Bluetooth, Wi-Fi, ANT/ANT+, ZigBee, etc.).

In addition, the UE device 301 can execute an application 401 that is a software client for storing, processing, and/or forwarding the sensor data to other components of the system 100. In one embodiment, the application 401 may include a sensor manager coordinating the collection and aggregation of biomarker data 101 as discussed with respect to the various embodiments of the approach described herein. In addition or alternatively, it is contemplated that the UE device 301 may include a standalone sensor manager that operates independently of the application 401, and that the sensors themselves may include a sensor manager.

By way of example, the communication network 307 of system 100 includes one or more networks such as a data network (not shown), a wireless network (not shown), a telephony network (not shown), or any combination thereof. It is contemplated that the data network may be any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network may be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UNITS), etc., as well as any other suitable wireless medium, e.g., worldwide interoperability for microwave access (WiMAX), Long Term Evolution (LTE) networks, code division multiple access (CDMA), wideband code division multiple access (WCDMA), wireless fidelity (Wi-Fi), wireless LAN (WLAN), Bluetooth®, Internet Protocol (IP) data casting, satellite, mobile ad-hoc network (MANET), and the like, or any combination thereof.

The UE device 301 is any type of mobile terminal, fixed terminal, or portable terminal including a mobile handset, station, unit, device, multimedia computer, multimedia tablet, Internet node, communicator, desktop computer, laptop computer, notebook computer, netbook computer, tablet computer, personal communication system (PCS) device, personal navigation device, personal digital assistants (PDAs), audio/video player, digital camera/camcorder, positioning device, television receiver, radio broadcast receiver, electronic book device, game device, or any combination thereof, including the accessories and peripherals of these devices, or any combination thereof. It is also contemplated that the UE device 301 can support any type of interface to the user (such as “wearable” circuitry, etc.).

By way of example, the UE device 301, data collection platform 103, group synchrony platform 109, and/or other components of the system 100 communicate with each other using well known, new or still developing protocols. In this context, a protocol includes a set of rules defining how the network nodes within the communication network 307 interact with each other based on information sent over the communication links. The protocols are effective at different layers of operation within each node, from generating and receiving physical signals of various types, to selecting a link for transferring those signals, to the format of information indicated by those signals, to identifying which software application executing on a computer system sends or receives the information. The conceptually different layers of protocols for exchanging information over a network are described in the Open Systems Interconnection (OSI) Reference Model.

Communications between the network nodes are typically effected by exchanging discrete packets of data. Each packet typically comprises (1) header information associated with a particular protocol, and (2) payload information that follows the header information and contains information that may be processed independently of that particular protocol. In some protocols, the packet includes (3) trailer information following the payload and indicating the end of the payload information. The header includes information such as the source of the packet, its destination, the length of the payload, and other properties used by the protocol. Often, the data in the payload for the particular protocol includes a header and payload for a different protocol associated with a different, higher layer of the OSI Reference Model. The header for a particular protocol typically indicates a type for the next protocol contained in its payload. The higher layer protocol is said to be encapsulated in the lower layer protocol. The headers included in a packet traversing multiple heterogeneous networks, such as the Internet, typically include a physical (layer 1) header, a data-link (layer 2) header, an internetwork (layer 3) header and a transport (layer 4) header, and various application headers (layer 5, layer 6 and layer 7) as defined by the OSI Reference Model.

In one embodiment, the application 401/UE device 301 and the group synchrony platform 109 may interact according to a client-server model. According to the client-server model, a client process sends a message including a request to a server process, and the server process responds by providing a service (e.g., providing map information). The server process may also return a message with a response to the client process. Often the client process and server process execute on different computer devices, called hosts, and communicate via a network using one or more protocols for network communications. The term “server” is conventionally used to refer to the process that provides the service, or the host computer on which the process operates. Similarly, the term “client” is conventionally used to refer to the process that makes the request, or the host computer on which the process operates. As used herein, the terms “client” and “server” refer to the processes, rather than the host computers, unless otherwise clear from the context. In addition, the process performed by a server can be broken up to run as multiple processes on multiple hosts (sometimes called tiers) for reasons that include reliability, scalability, and redundancy, among others.

The processes described herein for providing group synchrony based on biomarkers may be advantageously implemented via software, hardware (e.g., general processor, Digital Signal Processing (DSP) chip, an Application Specific Integrated Circuit (ASIC), Field Programmable Gate Arrays (FPGAs), etc.), firmware or a combination thereof. Such exemplary hardware for performing the described functions is detailed below.

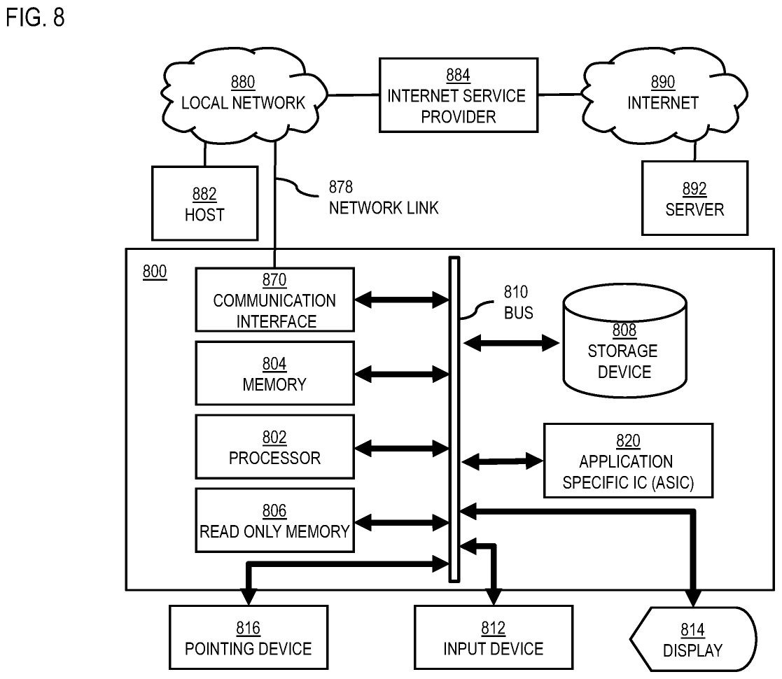

FIG. 8 illustrates a computer system 800 upon which an embodiment of the invention may be implemented. Computer system 800 is programmed (e.g., via computer program code or instructions) to provide group synchrony based on biomarkers as described herein and includes a communication mechanism such as a bus 810 for passing information between other internal and external components of the computer system 800. Information (also called data) is represented as a physical expression of a measurable phenomenon, typically electric voltages, but including, in other embodiments, such phenomena as magnetic, electromagnetic, pressure, chemical, biological, molecular, atomic, sub-atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range.

A bus 810 includes one or more parallel conductors of information so that information is transferred quickly among devices coupled to the bus 810. One or more processors 802 for processing information are coupled with the bus 810.

A processor 802 performs a set of operations on information as specified by computer program code related to providing group synchrony based on biomarkers. The computer program code is a set of instructions or statements providing instructions for the operation of the processor and/or the computer system to perform specified functions. The code, for example, may be written in a computer programming language that is compiled into a native instruction set of the processor. The code may also be written directly using the native instruction set (e.g., machine language). The set of operations include bringing information in from the bus 810 and placing information on the bus 810. The set of operations also typically include comparing two or more units of information, shifting positions of units of information, and combining two or more units of information, such as by addition or multiplication or logical operations like OR, exclusive OR (XOR), and AND. Each operation of the set of operations that can be performed by the processor is represented to the processor by information called instructions, such as an operation code of one or more digits. A sequence of operations to be executed by the processor 802, such as a sequence of operation codes, constitute processor instructions, also called computer system instructions or, simply, computer instructions. Processors may be implemented as mechanical, electrical, magnetic, optical, chemical or quantum components, among others, alone or in combination.

Computer system 800 also includes a memory 804 coupled to bus 810. The memory 804, such as a random access memory (RANI) or other dynamic storage device, stores information including processor instructions for providing group synchrony based on biomarkers. Dynamic memory allows information stored therein to be changed by the computer system 800. RAM allows a unit of information stored at a location called a memory address to be stored and retrieved independently of information at neighboring addresses. The memory 804 is also used by the processor 802 to store temporary values during execution of processor instructions. The computer system 800 also includes a read only memory (ROM) 806 or other static storage device coupled to the bus 810 for storing static information, including instructions, that is not changed by the computer system 800. Some memory is composed of volatile storage that loses the information stored thereon when power is lost. Also coupled to bus 810 is a non-volatile (persistent) storage device 808, such as a magnetic disk, optical disk or flash card, for storing information, including instructions, that persists even when the computer system 800 is turned off or otherwise loses power.

Information, including instructions for providing group synchrony based on biomarkers, is provided to the bus 810 for use by the processor from an external input device 812, such as a keyboard containing alphanumeric keys operated by a human user, or a sensor. A sensor detects conditions in its vicinity and transforms those detections into physical expression compatible with the measurable phenomenon used to represent information in computer system 800. Other external devices coupled to bus 810, used primarily for interacting with humans, include a display device 814, such as a cathode ray tube (CRT) or a liquid crystal display (LCD), or plasma screen or printer for presenting text or images, and a pointing device 816, such as a mouse or a trackball or cursor direction keys, or motion sensor, for controlling a position of a small cursor image presented on the display 814 and issuing commands associated with graphical elements presented on the display 814. In some embodiments, for example, in embodiments in which the computer system 800 performs all functions automatically without human input, one or more of external input device 812, display device 814 and pointing device 816 is omitted.

In the illustrated embodiment, special purpose hardware, such as an application specific integrated circuit (ASIC) 820, is coupled to bus 810. The special purpose hardware is configured to perform operations not performed by processor 802 quickly enough for special purposes. Examples of application specific ICs include graphics accelerator cards for generating images for display 814, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 800 also includes one or more instances of a communications interface 870 coupled to bus 810. Communication interface 870 provides a one-way or two-way communication coupling to a variety of external devices that operate with their own processors, such as printers, scanners and external disks. In general the coupling is with a network link 878 that is connected to a local network 880 to which a variety of external devices with their own processors are connected. For example, communication interface 870 may be a parallel port or a serial port or a universal serial bus (USB) port on a personal computer. In some embodiments, communications interface 870 is an integrated services digital network (ISDN) card or a digital subscriber line (DSL) card or a telephone modem that provides an information communication connection to a corresponding type of telephone line. In some embodiments, a communication interface 870 is a cable modem that converts signals on bus 810 into signals for a communication connection over a coaxial cable or into optical signals for a communication connection over a fiber optic cable. As another example, communications interface 870 may be a local area network (LAN) card to provide a data communication connection to a compatible LAN, such as Ethernet. Wireless links may also be implemented. For wireless links, the communications interface 870 sends or receives or both sends and receives electrical, acoustic or electromagnetic signals, including infrared and optical signals, that carry information streams, such as digital data. For example, in wireless handheld devices, such as mobile telephones like cell phones, the communications interface 870 includes a radio band electromagnetic transmitter and receiver called a radio transceiver. In certain embodiments, the communications interface 870 enables connection to the communication network 307 for providing group synchrony based on biomarkers.

The term computer-readable medium is used herein to refer to any medium that participates in providing information to processor 802, including instructions for execution. Such a medium may take many forms, including, but not limited to, non-volatile media, volatile media and transmission media. Non-volatile media include, for example, optical or magnetic disks, such as storage device 808. Volatile media include, for example, dynamic memory 804. Transmission media include, for example, coaxial cables, copper wire, fiber optic cables, and carrier waves that travel through space without wires or cables, such as acoustic waves and electromagnetic waves, including radio, optical and infrared waves. Signals include man-made transient variations in amplitude, frequency, phase, polarization or other physical properties transmitted through the transmission media. Common forms of computer-readable media include, for example, a floppy disk, a flexible disk, hard disk, magnetic tape, any other magnetic medium, a CD-ROM, CDRW, DVD, any other optical medium, punch cards, paper tape, optical mark sheets, any other physical medium with patterns of holes or other optically recognizable indicia, a RAM, a PROM, an EPROM, a FLASH-EPROM, any other memory chip or cartridge, a carrier wave, or any other medium from which a computer can read.

Network link 878 typically provides information communication using transmission media through one or more networks to other devices that use or process the information. For example, network link 878 may provide a connection through local network 880 to a host computer 882 or to equipment 884 operated by an Internet Service Provider (ISP). ISP equipment 884 in turn provides data communication services through the public, world-wide packet-switching communication network of networks now commonly referred to as the Internet 890.

A computer called a server host 892 connected to the Internet hosts a process that provides a service in response to information received over the Internet. For example, server host 892 hosts a process that provides information representing video data for presentation at display 814. It is contemplated that the components of system can be deployed in various configurations within other computer systems, e.g., host 882 and server 892.

FIG. 9 illustrates a chip set 900 upon which an embodiment of the invention may be implemented. Chip set 900 is programmed to provide group synchrony based on biomarkers as described herein and includes, for instance, the processor and memory components described with respect to FIG. 8 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip.

In one embodiment, the chip set 900 includes a communication mechanism such as a bus 901 for passing information among the components of the chip set 900. A processor 903 has connectivity to the bus 901 to execute instructions and process information stored in, for example, a memory 905. The processor 903 may include one or more processing cores with each core configured to perform independently. A multi-core processor enables multiprocessing within a single physical package. Examples of a multi-core processor include two, four, eight, or greater numbers of processing cores. Alternatively or in addition, the processor 903 may include one or more microprocessors configured in tandem via the bus 901 to enable independent execution of instructions, pipelining, and multithreading. The processor 903 may also be accompanied with one or more specialized components to perform certain processing functions and tasks such as one or more digital signal processors (DSP) 907, or one or more application-specific integrated circuits (ASIC) 909. A DSP 907 typically is configured to process real-world signals (e.g., sound) in real time independently of the processor 903. Similarly, an ASIC 909 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 903 and accompanying components have connectivity to the memory 905 via the bus 901. The memory 905 includes both dynamic memory (e.g., RAM, magnetic disk, writable optical disk, etc.) and static memory (e.g., ROM, CD-ROM, etc.) for storing executable instructions that when executed perform the inventive steps described herein to provide group synchrony based on biomarkers. The memory 905 also stores the data associated with or generated by the execution of the inventive steps.

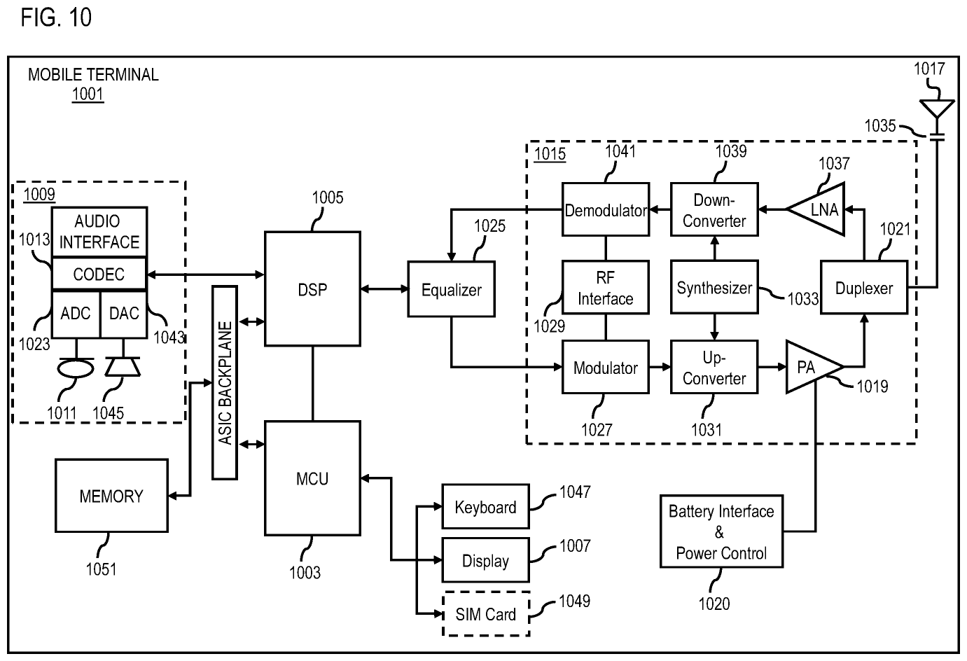

FIG. 10 is a diagram of exemplary components of a mobile terminal (e.g., handset) capable of operating in the system of FIG. 1, according to one embodiment. Generally, a radio receiver is often defined in terms of front-end and back-end characteristics. The front-end of the receiver encompasses all of the Radio Frequency (RF) circuitry whereas the back-end encompasses all of the base-band processing circuitry. Pertinent internal components of the telephone include a Main Control Unit (MCU) 1003, a Digital Signal Processor (DSP) 1005, and a receiver/transmitter unit including a microphone gain control unit and a speaker gain control unit. A main display unit 1007 provides a display to the user in support of various applications and mobile station functions that offer automatic contact matching. An audio function circuitry 1009 includes a microphone 1011 and microphone amplifier that amplifies the speech signal output from the microphone 1011. The amplified speech signal output from the microphone 1011 is fed to a coder/decoder (CODEC) 1013.

A radio section 1015 amplifies power and converts frequency in order to communicate with a base station, which is included in a mobile communication system, via antenna 1017. The power amplifier (PA) 1019 and the transmitter/modulation circuitry are operationally responsive to the MCU 1003, with an output from the PA 1019 coupled to the duplexer 1021 or circulator or antenna switch, as known in the art. The PA 1019 also couples to a battery interface and power control unit 1020.

In use, a user of mobile station 1001 speaks into the microphone 1011 and his or her voice along with any detected background noise is converted into an analog voltage. The analog voltage is then converted into a digital signal through the Analog to Digital Converter (ADC) 1023. The control unit 1003 routes the digital signal into the DSP 1005 for processing therein, such as speech encoding, channel encoding, encrypting, and interleaving. In one embodiment, the processed voice signals are encoded, by units not separately shown, using a cellular transmission protocol such as global evolution (EDGE), general packet radio service (GPRS), global system for mobile communications (GSM), Internet protocol multimedia subsystem (IMS), universal mobile telecommunications system (UNITS), etc., as well as any other suitable wireless medium, e.g., microwave access (WiMAX), Long Term Evolution (LTE) networks, 5G New Radio networks, code division multiple access (CDMA), wireless fidelity (WiFi), satellite, and the like.

The encoded signals are then routed to an equalizer 1025 for compensation of any frequency-dependent impairments that occur during transmission though the air such as phase and amplitude distortion. After equalizing the bit stream, the modulator 1027 combines the signal with a RF signal generated in the RF interface 1029. The modulator 1027 generates a sine wave by way of frequency or phase modulation. In order to prepare the signal for transmission, an up-converter 1031 combines the sine wave output from the modulator 1027 with another sine wave generated by a synthesizer 1033 to achieve the desired frequency of transmission. The signal is then sent through a PA 1019 to increase the signal to an appropriate power level. In practical systems, the PA 1019 acts as a variable gain amplifier whose gain is controlled by the DSP 1005 from information received from a network base station. The signal is then filtered within the duplexer 1021 and optionally sent to an antenna coupler 1035 to match impedances to provide maximum power transfer. Finally, the signal is transmitted via antenna 1017 to a local base station. An automatic gain control (AGC) can be supplied to control the gain of the final stages of the receiver. The signals may be forwarded from there to a remote telephone which may be another cellular telephone, other mobile phone or a land-line connected to a Public Switched Telephone Network (PSTN), or other telephony networks.

Voice signals transmitted to the mobile station 1001 are received via antenna 1017 and immediately amplified by a low noise amplifier (LNA) 1037. A down-converter 1039 lowers the carrier frequency while the demodulator 1041 strips away the RF leaving only a digital bit stream. The signal then goes through the equalizer 1025 and is processed by the DSP 1005. A Digital to Analog Converter (DAC) 1043 converts the signal and the resulting output is transmitted to the user through the speaker 1045, all under control of a Main Control Unit (MCU) 1003—which can be implemented as a Central Processing Unit (CPU) (not shown).

The MCU 1003 receives various signals including input signals from the keyboard 1047. The keyboard 1047 and/or the MCU 1003 in combination with other user input components (e.g., the microphone 1011) comprise a user interface circuitry for managing user input. The MCU 1003 runs a user interface software to facilitate user control of at least some functions of the mobile station 1001 to provide group synchrony based on biomarkers. The MCU 1003 also delivers a display command and a switch command to the display 1007 and to the speech output switching controller, respectively. Further, the MCU 1003 exchanges information with the DSP 1005 and can access an optionally incorporated SIM card 1049 and a memory 1051. In addition, the MCU 1003 executes various control functions required of the station. The DSP 1005 may, depending upon the implementation, perform any of a variety of conventional digital processing functions on the voice signals. Additionally, DSP 1005 determines the background noise level of the local environment from the signals detected by microphone 1011 and sets the gain of microphone 1011 to a level selected to compensate for the natural tendency of the user of the mobile station 1001.

The CODEC 1013 includes the ADC 1023 and DAC 1043. The memory 1051 stores various data including call incoming tone data and is capable of storing other data including music data received via, e.g., the global Internet. The software module could reside in RAM memory, flash memory, registers, or any other form of writable computer-readable storage medium known in the art including non-transitory computer-readable storage medium. For example, the memory device 1051 may be, but not limited to, a single memory, CD, DVD, ROM, RAM, EEPROM, optical storage, or any other non-volatile or non-transitory storage medium capable of storing digital data.

An optionally incorporated SIM card 1049 carries, for instance, important information, such as the cellular phone number, the carrier supplying service, subscription details, and security information. The SIM card 1049 serves primarily to identify the mobile station 1001 on a radio network. The card 1049 also contains a memory for storing a personal telephone number registry, text messages, and user specific mobile station settings.

While the invention has been described in connection with a number of embodiments and implementations, the invention is not so limited but covers various obvious modifications and equivalent arrangements, which fall within the purview of the appended claims. Although features of the invention are expressed in certain combinations among the claims, it is contemplated that these features can be arranged in any combination and order.

What is claimed is:

1. A method comprising:
receiving biomarker data collected from one or more sensors for a group of at least two users, wherein the biomarker data includes respective biomarker values for the at least two users, another group of other users, or a combination thereof;
computing a group synchrony score based on a metric indicating a similarity or a difference of the respective biomarker values; and
providing the group synchrony score as an output in a user interface of a device.

2. The method of claim 1, further comprising:
initiating a comparison of the group synchrony score, the respective biomarker values, the similarity, the difference, or a combination thereof to biomarker criteria associated with a reference group interaction,
wherein the output further includes the comparison.

3. The method of claim 1, wherein the group synchrony score is computed with respect to the group, to an individual user of the group, or a combination thereof.

4. The method of claim 1, further comprising:
determining a recommended action to be performed by the group, one or more of the at least two users, or a combination thereof to change the group synchrony score,
wherein the output further includes the recommended action.

5. The method of claim 1, further comprising:
recording the biomarker data as the group, one or more of the at least two users, or a combination thereof performs an activity; and
replaying the group synchrony score, the recorded biomarker data, or a combination thereof in association with a progress of the activity in the user interface of the device.

6. The method of claim 1, wherein the group synchrony score is computed and provided in real-time, asynchronously, or according to a schedule to update the output in the user interface.

7. The method of claim 1, further comprising:
receiving an input specifying an initial state of the group; and
generating an association between the group synchrony score and the initial state,
wherein the output further includes the association.

8. The method of claim 1, further comprising:
receiving an input specifying a target state, target interaction, or a combination thereof of the group; and
initiating a comparison of the group synchrony score to the target state, the target interaction, or a combination thereof,
wherein the output further includes the comparison.

9. The method of claim 8, further comprising:
determining a current activity in which the group is engaged; and
generating a prediction of whether the group will achieve the target state, target interaction, or a combination thereof based on the current activity and the group synchrony score.

10. The method of claim 9, wherein the prediction is performed using a machine learning model.

11. The method of claim 1, wherein the biomarker data is multi-dimensional with respect to one or more types of the biomarker data, the method further comprising:
receiving an input specifying an initial dimension of the multi-dimensional biomarker data,
wherein the group synchrony score is computed with respect to the initial dimension.

12. The method of claim 1, wherein the biomarker data includes heart rate data, respiratory rate data, electrical brain activity data, galvanic skin response data, neuroimaging data, accelerometer data, or a combination thereof.

13. The method of claim 1, wherein the biomarker data includes one or more social cues extracted from sensor data collected from the one or more sensors.

14. The method of claim 13, wherein the social cues include a facial expression, a movement, a contextual speech or conversation, a verbal cue, a non-verbal cue, or a combination thereof extracted from image data, audio data, or a combination thereof of the sensor data.

15. An apparatus comprising:
at least one processor; and
at least one memory including computer program code,
the at least one memory and the computer program code configured to, with the at least one processor, cause the apparatus to perform at least the following,
receive biomarker data collected from one or more sensors for a group of at least two users, wherein the biomarker data includes respective biomarker values for the at least two users, another group of other users, or a combination thereof;
compute a group synchrony score based on a metric indicating a similarity or a difference of the respective biomarker values; and
provide the group synchrony score as an output in a user interface of a device.

16. The apparatus of claim 15, wherein the apparatus is further caused to:
initiate a comparison of the group synchrony score, the respective biomarker values, the similarity, the difference, or a combination thereof to biomarker criteria associated with a reference group interaction,
wherein the output further includes the comparison.

17. The apparatus of claim 15, wherein the group synchrony score is computed with respect to the group, to an individual user of the group, or a combination thereof.

18. A computer-readable storage medium carrying one or more sequences of one or more instructions which, when executed by one or more processors, cause an apparatus to perform at least:
receiving biomarker data collected from one or more sensors for a group of at least two users, wherein the biomarker data includes respective biomarker values for the at least two users, another group of other users, or a combination thereof;
computing a group synchrony score based on a metric indicating a similarity or a difference of the respective biomarker values; and
providing the group synchrony score as an output in a user interface of a device.

19. The computer-readable storage medium of claim 18, wherein the apparatus is caused to further perform:
initiating a comparison of the group synchrony score, the respective biomarker values, the similarity, the difference, or a combination thereof to biomarker criteria associated with a reference group interaction,
wherein the output further includes the comparison.

20. The computer-readable storage medium of claim 18, wherein the group synchrony score is computed with respect to the group, to an individual user of the group, or a combination thereof.
